(19) United States
(12) Patent Application Publication
(10) Pub. No.: US 2013/0203171 A1
(43) Pub. Date: Aug. 8, 2013

(54) ANALYTICAL METHOD AND TITRATION DEVICE

(71) Applicant: John Richard Sportsman, Encinitas, CA (US)

(72) Inventor: John Richard Sportsman, Encinitas, CA (US)

(73) Assignee: SPORTSMAN CONSULTING LLC, Carlsbad, CA (US)

(21) Appl. No.: 13/879,093

(22) PCT Filed: Oct. 14, 2011
(86) PCT No.: PCT/US11/56232

§ 371 Date: Apr. 12, 2013

Related U.S. Application Data
(60) Provisional application No. 61/455,140, filed on Oct. 14, 2010.

(51) Int. Cl. G01N 33/14

(52) U.S. Cl. CPC G01N 33/146
USPC 436/24; 436/20

(57) ABSTRACT

A device and reagents to be used in conjunction therewith for the determination of the level of sulfite or related species, including sulfur dioxide (SO₂), in red or white wine, musts, beer, juices, water, industrial process streams and other opaque media is described. The device provides electrodes and circuits designed for amperometric detection of the titration endpoint and for indicating by sight, sound or touch when the endpoint has been reached. The reagents allow quantitative volumetric determination of the iodometric endpoint when used with the device. The sulfite or related species levels are related directly to the volume of the quantitative reagents used.

### RELATED APPLICATION

This application claims the benefit of U.S. provisional application No. 61/455,140, filed 14 Oct. 2010, the entire disclosure of which is incorporated herein by reference.

### FIELD OF THE INVENTION

The present invention is in the technical field of chemical analysis. In particular, the present invention relates to analysis of sulfite and related forms such as bisulfite and sulfur dioxide (SO₂). More particularly, the present invention is in the area of analysis of sulfite by iodometric titration.

### BACKGROUND

Sulfites are antioxidants that are sometimes added to foods and beverages to prevent spoilage and preserve freshness. They also present a known health risk, causing in some instances headaches and allergic responses. Aside from the benefits and risks associated with sulfite use in foods and beverages sulfites are important in many industrial processes including water treatment, surface water monitoring, and paper production. There has been a long felt need for an inexpensive, accurate method of determining sulfite levels in water, wine, must, beer, wort, juices and other opaque liquids, suspensions or other media.

Sulfites come in various forms, such as ions of sulfite (SO₃²⁻), bisulfite (HSO₃⁻), or metabisulfite (S₂O₅²⁻), salts of sulfite, bisulfite or metabisulfite (e.g., potassium sulfite, sodium bisulfite) and related species like sulfur dioxide (SO₂). Aqueous solutions of sulfur dioxide are sometimes referred to as sulfurous acid.

In wine making, sulfite is added, typically as potassium metabisulfite, to wine or must to control spoilage and the fermentation process as well as to prevent oxidation of the wine. In solution, sulfite will be in a pH and temperature dependent equilibrium with sulfur dioxide. Sulfite and sulfur dioxide protect wine from not only oxidation, but also bacteria. Without sulfur, grape juice would turn to vinegar. Maintaining an optimal sulfur dioxide level can prevent the discoloration, off-flavors and unpleasant aromas resulting from oxidation and microbial contamination. However, sulfur dioxide levels are continually reduced as it protects the wine. Therefore it is important to know whether and when to add sulfite during the wine making process.

In the U.S.A., wine with sulfite content at or above 10 ppm (10 mg/liter of SO₂) must be labeled as containing sulfites. The legal maximum sulfite content for wine is 350 ppm, while most wines average about 80 ppm.

The determination of sulfite levels in food, beverages, and industrial process streams is important to food safety, human health and control of important industrial processes. Typically such analysis is accomplished by titration methods, generally comprising addition of measured volumes of a titrant of known concentration that reacts with the sulfite, or with a preformed derivative of the sulfite, until some quantitative endpoint is reached.

In one approach, a liquid sample containing sulfite to be analyzed is acidified, and then subjected to an aeration process where the SO₂ in the sample is swept into a capture solution containing hydrogen peroxide. The peroxide oxidizes the sulfite (SO₂) to sulfate (SO₃²⁻), which is then titrated as sulfuric acid (H₂SO₄) by standard sodium hydroxide (NaOH) to a pH of 9. In this method, called aeration-oxidation or Monier-Williams, the volume of NaOH can be related to the amount of SO₂ originally present in the sample. The modern version of this procedure is described by J. B. Thompson, and E. Toy, “Determination of Sulfur Dioxide. Improved Monier-Williams Method” Ind. Eng. Chem. Anal. Ed., 17: 612-615 (1945).

The aeration-oxidation method can be done by hand, but it is time-consuming, suffers from incomplete recovery of the sulfur dioxide and subject to errors of operation even using modern procedures. Each test requires a recovery step followed by a titration with an accurate pH meter.

Another approach is the Ripper titration, described by M. Ripper in 1898, in which a liquid sample containing sulfite to be analyzed is acidified, and then titrated with standardized oxidant, specifically, iodine or iodate solution. This is a procedure called iodometric titration and is practiced in modern form largely as described by M. A. Joslyn, “Sulfur Dioxide Content of Wine by Iodometric Titration” Am. J. Enol. Vitic. 6: 1-10 (1955).

The Ripper titration is based on the quantitative reaction of sulfur dioxide with iodine (iodate solutions generate iodine) which oxidizes the sulfur dioxide in the sample under acid conditions. The pertinent chemical reactions are:

IO₃⁻+5I⁻+6H⁺→3I₂+3H₂O generation of iodine from iodate

SO₂+I₂+H₂O→2I⁻+SO₃+2H⁺ reaction of SO₂ and iodine

The endpoint of the Ripper titration can be detected by adding a starch solution to the sample prior to reaching the endpoint. When the endpoint is reached, excess iodine appears and complexes with the starch, forming a deep blue color—a highly visual indication of the endpoint. Free sulfite is determined directly while total sulfite can be ascertained by treating the sample with sodium hydroxide (before acidifying and titrating) to release bound sulfites.

Ripper titrations have the advantage that they are fast and easy to run. Unfortunately Ripper titrations suffer from serious problems owing to the visual indication of the endpoint in the sample. The endpoint is subject to errors of interpretation and very difficult to see in opaque media—media where any combination of absorption and scattering reduces the direct transmission of at least some visible wavelengths of light. Many economically important samples such as wine, must, beer, wort, and juice (fruit or vegetable) and industrial process streams are significantly opaque. Even waste water and natural waters may be opaque enough to interfere with reliable visualization of the starch-indicated endpoint.

To overcome interference from opaque solutions, one can dilute the sample but this will lessen the sensitivity at the endpoint, as noted by L. Benvegnin and E. Capt, “Du dosage de l'acide sulfureux libre et total dans les vins rouges” Mitt. Gebiete Lebensm. u. Hyg. 22, 257-63, 365-8 (1931).

Some modifications to the Ripper method seek to improve the reliability of detecting the endpoint. For example, the use of electrochemical sensors that respond to the appearance of free iodine removes the burden of trying to visualize the starch endpoint against a colored background in opaque media. J. A. Vail and J. B. Converse, in J. Assoc. Off. Anal. Chem. 63:1, 194-199, describe the use of the Ripper method, including the use of an electronic pH meter set up for Karl Fischer (dead stop) titration using a potentiometric endpoint. In effect, the paper describes the measurement of the oxidation potential of the solution at the endpoint. The paper recommends that the standard Ripper technique not be used for sulfite determination in wine because of severe problems in determining the starch iodine endpoint. Joslyn (cited above) also reported that use of electrodes like this do not improve the precision of the method.

In another potentiometric measurement, the Ripper method endpoint is detected with an oxidation-reduction probe (ORP). The ORP is sensitive to the change in oxidation status of the solution that occurs as free iodine appears at the endpoint. Using an ORP system partly solves the problem of color interference, but the ORP potential change is relatively small at the endpoint, requiring over-titration and back-extrapolation to determine the endpoint. For this reason, almost all ORP systems require expensive titrant delivery systems and microprocessor-based calculations to be useful, raising the cost of these systems above the average winery's budget for analysis.

Detection of iodine in iodometric titrations has also been performed by amperometric methods. The use of dual platinum electrodes for detecting iodine was described as early as 1906 by E. Brunner, Z. Physik. Chem. 56, 321 and it is described as used in a titrimetric method in 1974 by Willard, Merritt and Dean, Instrumental Methods of Analysis, 5th ed., D. Van Nostrand Co., New York, pp. 734-9. In this method, platinum electrodes are poised at a small voltage (typically 0.1 V), and any current between them is detected by a suitable electrometer. In the absence of iodine, there is little current; when iodine appears at the endpoint, current can be detected. Typically, the changes in current are small and, as in the case of the ORP electrode, the endpoint must therefore be estimated by over-titration and back-extrapolation.

Other sources of error with the Ripper method include the freeing of bound sulfur dioxide during the acidification step and the presence of other oxidizable compounds in the sample. If a sample contains bound and free sulfur dioxide, the acidification step (also required in some other methods like aeration-oxidation) induces slow dissociation of bound sulfur dioxide to form additional free sulfur dioxide, leading to a systematic overestimation of free sulfur dioxide.

Sulfur dioxide can also be overestimated if there are other oxidizable compounds (reducing agents) in the sample. These can react with the iodine titrant instead of the sulfur dioxide, again leading to overestimation of the sulfur dioxide level. For example, in some white wines, there are appreciable levels of ascorbic acid which react readily with the titrant. In red wines, phenolic compounds, like polyphenols, anthocyanin and pigments, can react with the titrant. The error induced by background reducing agents may be estimated by measuring the apparent sulfur dioxide with the Ripper method in a duplicate sample treated with hydrogen peroxide. Hydrogen peroxide specifically reacts with and removes all free sulfur dioxide from the sample. The difference between untreated and treated samples presents the true measure of sulfur dioxide in the sample, uncontaminated by background reactions.

### SUMMARY OF THE INVENTION

The present invention overcomes prior limitations in measuring sulfite or related species in fluid media. In particular, the disclosed instrument, reagents and methods of use enable rapid, accurate and sensitive analysis of sulfite and related species in fluid samples, especially opaque samples, such as water, wine, must, beer, wort, juice and industrial process streams. Though utilizing the basic Ripper titration system, the present invention does not rely on a sample color change to indicate the titration endpoint, eliminating guesswork in opaque samples. Instead, the present invention uses an amperometric detection system that indicates the titration endpoint. Upon indication of the titration endpoint, titration may be ceased, eliminating the need for over titration and back extrapolation of the endpoint.

The measuring device described herein is capable of applying a small voltage across poles of an electrode and measuring the increase in current detected by the electrode as the titration occurs and the concentration of iodine increases to the endpoint. At the endpoint there is a jump in iodine concentration and concomitant jump in electrode current. When the current rises above a predefined threshold, the user is signaled that the titration has reached its endpoint by an indicator mechanism, such as a light signal, audible tone or vibration.

### BRIEF DESCRIPTION OF THE DRAWINGS

Preferred and alternative embodiments are described in detail below with reference to the following drawings:

FIG. 1 is a schematic view of an exemplary titration system.

FIG. 2 is a cross sectional view of an exemplary electrode.

FIG. 3 is an exemplary schematic of a device for carrying out amperometric detection.

FIG. 4 is a process diagram depicting an exemplary method for preparing and titrating a test fluid.

### DETAILED DESCRIPTION OF THE INVENTION

The present invention uses a modified Ripper titration method with amperometric endpoint detection. The Ripper titration is based on the quantitative reaction of sulfur dioxide with iodine which oxidizes the sulfur dioxide in the sample under acid conditions. When all the sulfur dioxide is titrated at the endpoint, excess iodine appears in solution. This is detected as current with an electrode and signaled by visual, audible or tactile indicators. The endpoint is much more consistent and reliable than the starch endpoint employed for standard Ripper titrations. It is sharp and clear, even when titrating opaque fluids such as red wines and musts. From the known concentration of the titrant and its volume required to reach the endpoint, the free sulfur dioxide is simply calculated.

In the basic method, one starts with a sample that may contain sulfite or a related species such as sulfur dioxide. The sample may be a liquid, solution, suspension or some other fluid. The sample may be composed of significant fractions of solids or gases, but preferably the sample has a low enough viscosity such that mixing is not too cumbersome. Common samples include water (for water treatment or monitoring applications), food and beverages (for health and safety applications) and industrial process streams (for process control or environmental monitoring applications). The present invention is particularly advantageous for samples that are opaque to some degree, such as wine, must, beer, wort, and juices (including fruit and vegetable juices).

A known or determinable amount of the sample is prepared for titration by adding one or more substances and contacting with an electrode. The amount of sample can be determined by measuring the weight or volume using conventional devices such as buret or scale. The amount of sample can also be determined by prior knowledge or by placing a portion of the sample into a container of known or measurable volume. Typically one removes a small portion of the total sample to prepare a sample for testing.

One substance added to the known or determinable amount of sample is an acid or other component that will result in an acidified sample after addition. The most convenient substances are strong non-oxidizing acids in liquid form. For example hydrochloric acid (HCl) and sulfuric acid (H₂ SO₄) are suitable strong non-oxidizing acids. Other substances such as acid salts (e.g., sodium bisulfate, NaHSO₄) and weak non-oxidizing acids (e.g., citric acid, sulfamic acid and glycine-HCl) in solid or solution form are also suitable. Substances are suitable primarily when they are non-oxidizing and are used in sufficient quantity to make the sample under test sufficiently acidic. Non-oxidizing substances do not remove a significant amount of sulfite or related species from the test sample. The sample is sufficiently acidic when its pH is less than about 2. However iodometric titration is much more efficient at even lower pH. Preferably the substance added lowers the pH of the sample under test to less than 1. Wine typically has a pH of 3-4 and so requires acidification to speed the titration process. Some samples may be sufficiently acidic and would not require any acidification.

Another substance that may be added to the known or determinable amount of sample is a substance that will result in a significant amount of iodide. A significant concentration of iodide is convenient for iodometric titrations because the iodide ion helps to solubilize molecular iodine that is commonly used as the titrant. The iodide concentration is significant when it exceeds about 1 mM (millimolar), which is equivalent to a 0.02% wt/v solution of potassium iodide (KI). More preferably the iodide concentration after addition is greater than about 10 mM (equivalent to 0.2% wt/v KI). The substance added may be in liquid or solid form. If in solid form it is preferable to use a highly soluble compound like sodium iodide (NaI) or potassium iodide.

The substances, if any, to add to the known or determinable amount of sample may be added in any order. They may be added before or after determining the amount of the sample for testing. Moreover the substance to add acidity and the substance to add iodide may be the same (for example hydrogen iodide, HI), necessitating only one addition for both functions. Further, the substances added may comprise stabilizing components such as a buffer like sodium bicarbonate (NaHCO₃).

In addition to the optional additions of compounds to add acidity and to add iodide, the known or determinable amount of sample needs to be in electrical contact with an amperometric electrode. Electrical contact can be achieved by physical contact of the electrode and the sample, for example dipping the electrode into the known or determinable amount of sample, or by indirect methods such as a salt bridge or inductive coupling. The electrode may be placed in contact with the solution before, after or in conjunction with the additions.

The electrode necessarily has two or more poles. It is these poles that must be in contact with the sample. These poles are capable of conducting electricity and typically comprise relatively inert materials. Suitable materials for the poles include graphite, noble metals and alloys (e.g., platinum, gold, iridium, palladium, silver, osmium, and rhodium) and corrosion resistant metals or alloys (e.g., titanium, stainless steel). Poles may also be composed of a base material coated with a relatively inert material. Each pole may be composed of different materials or all poles may be composed of identical materials. Identical materials are preferable because it minimizes the electrode potential between the poles.

Convenient forms of the poles are wires, ribbons, rings, foils and sheets. Each pole may be a different form. The minimum cross sectional dimension for the poles is preferably greater than about 0.2 mm. The poles are spaced a short distance apart. The typical minimum spacing between the portions of the poles in contact with the sample is from 0.1-2 cm. Preferably the minimum spacing is less than about 1 cm.

The electrode is also connected to a device for measuring current between the poles. The device may be connected via electrical cable, wirelessly or may be integral with the electrode. The device is fundamentally an electrometer or ammeter that is capable of sensing a current as low as about 1 nA. The device may also be capable of detection modes other than amperometric. For example, the device may measure pH, temperature or other ion species when connected to suitable probes (pH, thermocouple, and ORP respectively).

At the titration endpoint the unreacted iodine will produce a current of approximately 10-100 nA. Besides detecting this current, the device should also be capable of impressing a small voltage between the poles. The voltage preferably is less than 0.5 V and more preferably is about 0.1 V. Alternatively or in conjunction with the impressed voltage, the poles may be dissimilar materials with an appropriate electrode potential difference.

Once the sample has been prepared for testing by adding the acidifying substance (if any), the iodide increasing substance (if any) and contacting the electrode, titration can begin. The titrant is typically an oxidant that is a solution of iodine or iodate. Molecular iodine does not dissolve into aqueous solutions easily. The presence of iodide ions improves the solubility of iodine. Iodide can be present in the sample, the sample as prepared with an iodide increasing substance, or in the titrant. Alternatively or in addition, an iodate solution may be part of the titrant. Under acid conditions and a slight excess of iodide, iodate solutions generate iodine. If the titrant comprises iodate, excess iodide can be present in the sample, the sample as prepared with an iodide increasing substance, or in the titrant.

Preferably the concentration of the titrant is in the range of the expected concentration of the sulfite or related species to be determined. For example, the limit for sulfite labeling of wine in the U.S.A. is 10 ppm SO₂, or 156 μM (micromolar). Convenient concentrations of iodine in the titrant are about 10 to 1,000 times higher, about 1-500 mM. These concentrations are convenient because the volume of titrant required to neutralize the sulfite or related species in the prepared sample is similar to or less than the volume of the prepared sample. The precise concentration of iodine in the titrant is not important as long as it is known or determinable. One way to know the concentration is to measure the ingredients used to prepare the titrant. One way that the concentration is determinable is by titrating the titrant against a known standard (standardizing), before or after use in the present invention.

To titrate the prepared sample, the titrant is added in a quantitative manner. The volume of titrant required to reach the endpoint can be used to determine the amount of sulfite or related species present in the sample. Therefore the titrant should be added in a controlled manner that allows for determination of the amount used. The titrant should be added slowly enough and in small enough quantities that the mixture can reach equilibrium. Commonly, titrant is added one drop at a time while agitating or stirring the prepared sample. Titrant may also be added at a slow continuous rate. The concentration of the titrant and the expected concentration of sulfite or related species guide the rate and volume of the titrant addition.

When sufficient titrant has been added, all of the sulfite or related species in the prepared sample will be reacted. This is the endpoint of the titration. Further addition of the titrant will add unreacted iodine which can be measured with the amperometric electrode and measuring device. The endpoint is signaled by an increase in the current between the poles due to excess iodine. The endpoint will yield a current of about 10-100 nA. Similarly, the endpoint is detectable when the iodine concentration rises above about 1 μM. The device compares the sensed current to a predetermined threshold, preferably in the range of 10-100 nA, and most conveniently at about 50 nA.

The measuring device is configured to indicate when the sensed current is above the predetermined threshold. The indication is a signal that the endpoint has been reached. The device may indicate visually such as with a lamp or LED that turns on, off, flashes or changes color. The device may also indicate with an audio signal such as with a speaker that starts or stops a tone, chirp, buzz or noise. Also the device may indicate in a tactile manner by starting or stopping vibrations, or moving a mechanical element.

When the measuring device indicates the endpoint has been reached, it is advantageous to stop adding titrant. Further titrant will not contribute to a more accurate determination of the sulfite or related species concentration. Any titrant expended after the endpoint would have to be accounted for when determining the volume of titrant necessary to react with all of the sulfite or related species in the prepared sample.

The object of the methods, reagents and apparatuses above is to measure or determine the concentration of sulfite or related species in the sample. The amount of sulfite or related species is determined from the amount of sample used, the amount of titrant used and the concentration of the titrant. The concentration of the titrant times the amount used is the quantity of iodine expended in mass or moles. In the quantitative reaction of sulfur dioxide and iodine, given in paragraph [0013], iodine and sulfur dioxide react in equal molar quantities. Therefore the number of moles of iodine expended is the number of moles of sulfur dioxide originally present in the sample tested. The concentration of the sulfur dioxide is then determined by dividing the calculated quantity of sulfur dioxide by the volume of the sample tested.

Besides being convenient and facile with opaque fluids, the present invention also suppresses errors commonly associated with Ripper titrations. The error reported due to the slow dissociation of bound sulfur dioxide after the acidification step can be minimized by performing the method of the present invention quickly. If the method is completed within two minutes, the dissociation of bound sulfur dioxide appears to contribute less than a few percent error in the free sulfur dioxide determination. The error reported due to background reducing agents in red wines (phenolic compounds and the like), is not typically seen when using the method of the present invention. The values determined with the method of the invention are not significantly different than what is determined with the aeration oxidation method, a method not affected by background reducing agents. Although it seems to be generally accepted that Ripper methods produce higher values and are less accurate than other methods, the present invention may attenuate such effects because of the improved reliability of detecting the endpoint. Table I presents a comparison of sulfur dioxide values in wine obtained with the present invention (New Method) and with the aeration-oxidation method (Std. Method).

TABLE I

| | New method, | Std. method, |
|---|---|---|
| Wine | ppm | ppm |
| Cabernet Sauvignon | 9 | 9 |
| Chardonnay | 16 | 15 |
| Malbec | 6 | 8 |
| Merlot | 4 | 2 |
| Petite Syrah | 17 | 15 |
| Red blend | 8 | 8 |
| Sangiovese | 13 | 12 |
| Zinfandel | 24 | 24 |

The present invention can be further understood by reference to the figures and following exemplary embodiments.

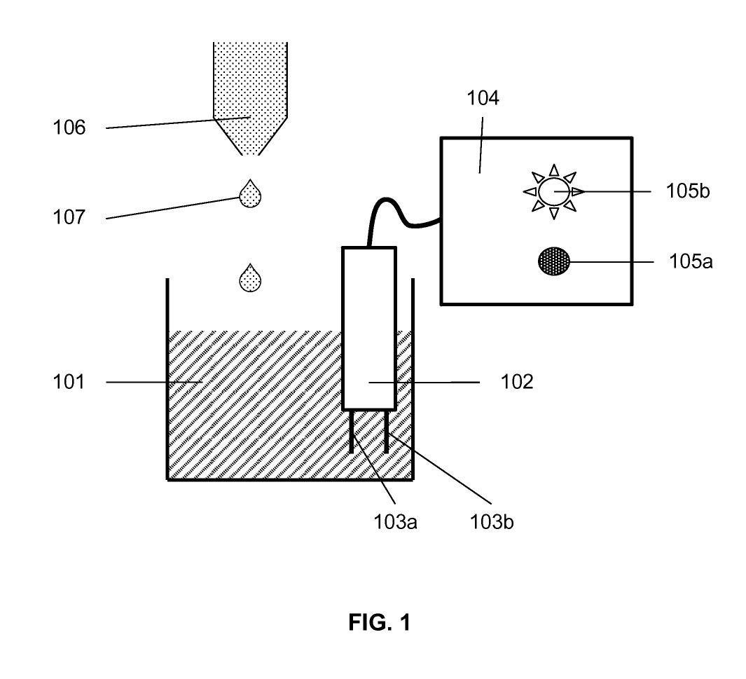

FIG. 1 is a schematic view of an exemplary titration system. The system includes a fluid test sample 101 that has either a known volume or has a determinable volume. The sulfite or related species concentration of the test sample can be determined by adding titrant 106, by drops 107 or otherwise, to the test sample 101. The titrant 106 comprises iodine or iodate. The electrode 102 comprises two poles 103a, 103b that are used by the current measuring device 104 to sense an excess current in the test sample 101 when the titration endpoint is reached. The measuring device 104 comprises circuitry to detect currents in the range of 1-1000 nA between the poles 103a and 103b and comprises circuitry to establish a voltage of about 0.05-0.5 V, preferably 0.1 V, between the poles 103a and 103b. The device 104 also is designed to indicate if the current between the poles 103a and 103b is greater than a set threshold by enabling the ‘stop’ indicator 105a. If the current is below the set threshold the device can enable the ‘proceed’ indicator 105b. When the ‘proceed’ indicator 105b is enabled, the user of the titration system knows to continue adding titrant 106. When the ‘stop’ indicator 105a is enabled, the user of the titration system knows to discontinue adding titrant because the endpoint has been achieved. The indicators 105a and 105b may be visual, audible or tactile in nature. When enabled, they may turn on or off; they may flash, beep, chirp, hum or vibrate.

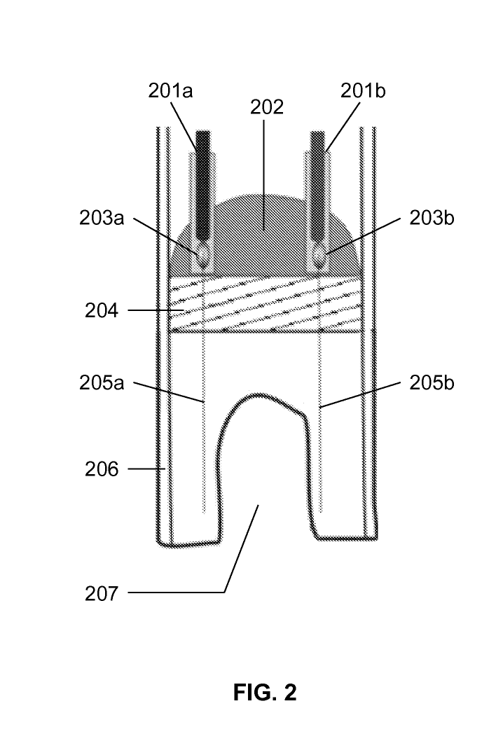

FIG. 2 is a cross sectional view of an exemplary electrode 102. The two poles 205a and 205b are connected via connections 203a and 203b respectively to two lead wires 201a and 201b respectively. Poles 205a and 205b commonly comprise inert conductive materials such as graphite, noble metals (including platinum, palladium and gold) and corrosion resistant materials (e.g. titanium). The connections 203a and 203b are commonly solder connections but may be any reasonable electrical connection including crimps. The lead wires 201a and 201b are commonly insulated copper conductors, although just about any high conductivity material would suffice. The lead wires 201a and 201b are electrically connected to the measuring device 104.

The poles 205a and 205b are exposed to the test sample 101 when the electrode 102 is in contact with the test sample 101. The lead wires 201a and 201b and connections 203a and 203b are protected from the test sample 101 by an inert, non-conductive electrode housing 206, an elastic bushing 204 and a sealant 202. The electrode housing 206 is commonly a glass tube open to the test sample 101 on the end housing the poles 205a and 205b. Additionally the electrode housing 206 may include a cutout region 207 to allow test sample 101 to circulate more freely to the poles 205a and 205b.

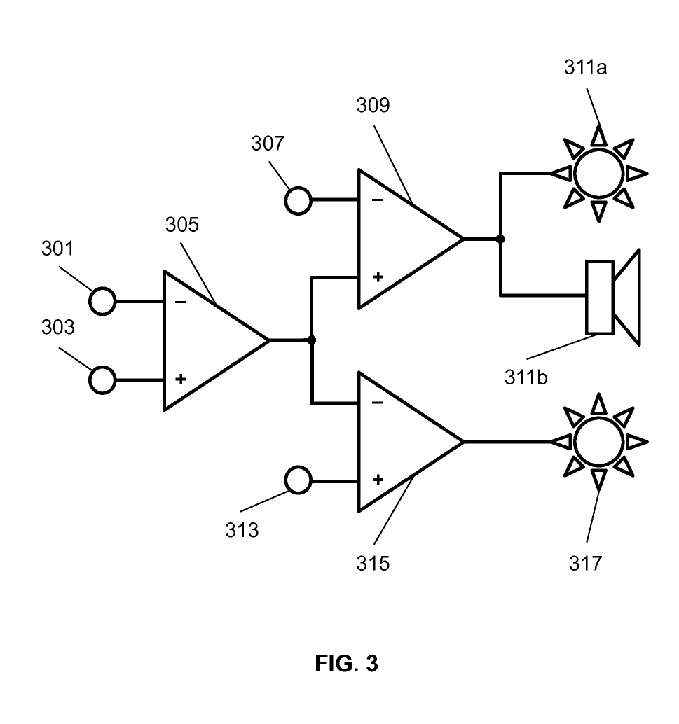

FIG. 3 is an exemplary schematic of a device 104 for carrying out amperometric detection. The electrode 102 is electrically connected to the probe input 301 via one of the poles 103a. The other pole 103b is referenced to ground. The probe input 301 is amplified and buffered by a probe amplifier 305. The second input to the probe amplifier 305 is a probe reference voltage 303. The probe amplifier 305 impresses the probe reference voltage 303 on the probe input 301 and thus across the electrode poles 103a and 103b. The probe reference voltage 303 is preferably between 0.05 V and 0.5 V, most preferably 0.1 V.

The amplified probe signal exits the probe amplifier 305 and serves as the positive input for a ‘stop’ comparator 309. The negative input for the ‘stop’ comparator 309 is a predetermined ‘stop’ threshold 307. The ‘stop’ threshold 307 corresponds to the amount of current in the test sample 101 when the titration reaches the endpoint. The ‘stop’ threshold 307 may correspond to about 10-100 nA, most conveniently it corresponds to 50 nA. The ‘stop’ threshold 307 may also correspond to an iodine concentration of 1 μM in the test sample 101. The ‘stop’ comparator 309 outputs a signal to enable the ‘stop’ indicators 311a and 311b if the amplified probe signal is greater than the ‘stop’ threshold 307. If the amplified probe signal is less than the ‘stop’ threshold 307, the ‘stop’ comparator 309 outputs a signal to disable the ‘stop’ indicators 311a and 311b. The ‘stop’ indicators 311a and 311b operate in the same way as the ‘stop’ indicator 105a.

The amplified probe signal also serves as the negative input for a ‘proceed’ comparator 315. The positive input for the ‘proceed’ comparator 315 is a predetermined ‘proceed’ threshold 313. Like the ‘stop’ threshold 307, the ‘proceed’ threshold 313 corresponds to the amount of current in the test sample 101 when the titration reaches the endpoint. The ‘proceed’ threshold 313 may be identical to or slightly lower than the ‘stop’ threshold 307. The ‘proceed’ comparator 315 outputs a signal to enable the ‘proceed’ indicator 317 if the amplified probe signal is less than the ‘proceed’ threshold 313. If the amplified probe signal is greater than the ‘proceed’ threshold 313, the ‘proceed’ comparator 315 outputs a signal to disable the ‘proceed’ indicator 317. The ‘proceed’ indicator 317 operates in the same way as the ‘proceed’ indicator 105b.

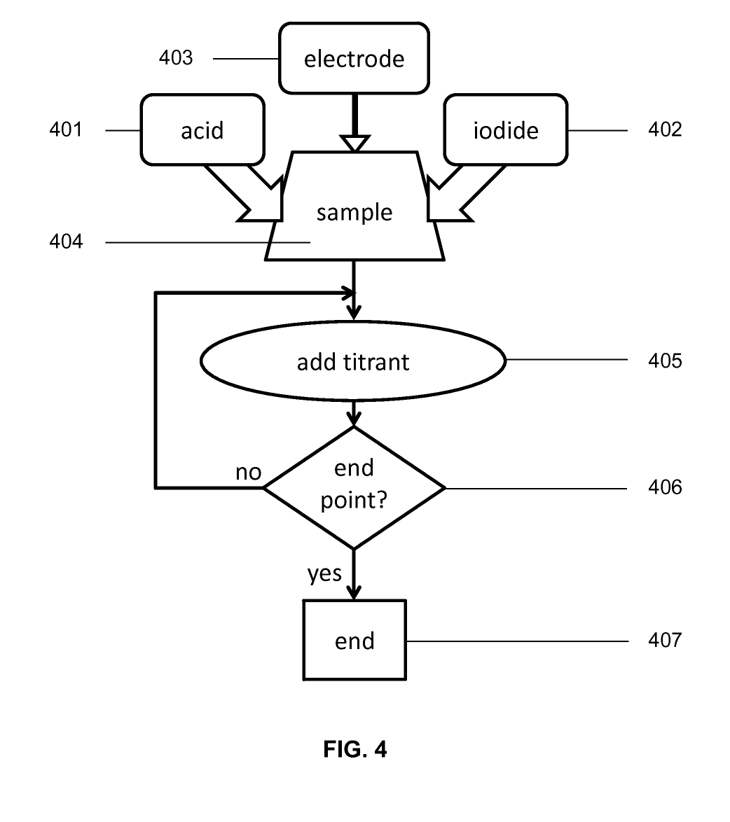

FIG. 4 is a process diagram depicting an exemplary method for preparing and titrating a test fluid. To a known or determinable volume of the sample 404, an acidifying substance 401 is added, an iodide increasing substance 402 is added, and an electrode 403 is contacted. The additions and contact may occur in any order. The sample 404 is a solution, suspension or other fluid media that may contain sulfites or related species. The acidifying substance 401 is a substance that causes the sample 404 to become acidic after addition. Preferably the sample pH is lowered below 2. The acidifying substance 401 may be a non-oxidizing acid or acid salt in liquid or solid form. Preferable acids include hydrochloric acid, sulfuric acid and citric acid. The iodide increasing substance 402 causes the sample 404 to increase in iodide after addition. Preferably the sample iodide concentration is increased to greater than 1 mM. The iodide increasing substance 402 may be salt of complex of iodide, such as potassium iodide and sodium iodide.

The electrode 403 that contacts the sample 404 comprises two poles. The electrode 403 is also electrically connected to a current sensing device such as the device 104. The current sensing device is capable of sensing the current between the two poles, impressing a voltage between the two poles, and indicating when the current between the two poles is greater than a predetermined threshold.

Once the sample 404 is prepared by adding an acidifying substance 401, adding an iodide increasing substance 402, and contacting with an electrode 403, titration may begin. Titration occurs by adding titrant 405 to the prepared sample. The titrant comprises a known or determinable amount of oxidant that will quantitatively react with the sulfite or related species that may be in the sample 404. Preferred titrants comprise iodine or iodate. The titrant is added quantitatively, i.e., measuring or determining the amount used is possible. The titrant is added to the prepared sample in small enough quantity and slowly enough to establish equilibrium in the mixture. The addition may be by drops or may be essentially continuous. Equilibrium may be assisted by swirling or agitating the mixture. As equilibrium is established for each drop or equivalent moment, the endpoint is checked by sensing the current in the electrode 406. If the current is below the predetermined threshold, addition of titrant 405 may be continued. If the current is above the predetermined threshold, the endpoint has been reached and the titration should stop 407. Once the endpoint has been reached, the sulfite or related species concentration may be calculated from the known or determined volume of the sample, the known or determined concentration of the titrant, and the volume of the titrant expended.

In one exemplary procedure for testing sulfites in wine or must, the following items are used: 1) current measuring device 104 with a green proceed LED 105b and a red stop LED and buzzer 105a, 2) electrode 102, 3) titration vessel, 100 mL beaker, 4) titrant dispenser, 5 mL syringe, and 5) distilled water in a wash bottle or similar. Additionally the following reagents are used: 1) reactant solution, 5% wt/v KI, 2) acid reagent, 2 N HCl, 3) titrant solution, 0.0156 N KIO₃, 4) optional base reagent, 1 N NaOH for total sulfite analysis.

To analyze free sulfur dioxide, first fill the syringe by drawing up the titrant solution. Expel bubbles and set the plunger on the syringe to a readable point, preferably the 5.0 mL point.

Second, place precisely 25 mL wine or must in the titration vessel. A 25 mL serological pipette can assist with accurate transfer.

Third, add approximately 2 mL acid reagent and 2 mL reactant solution to the titration vessel.

Fourth, rinse the electrode 102 briefly with distilled water. Insert the electrode into the titration vessel so that the tip is completely submerged to just above the circulation gaps 207 (cutouts at the tip of the electrode).

Fifth, begin stirring the mixture, using a constant moderate swirling motion. Hold the electrode 102 against the side of the vessel with one finger and grasp the vessel with the remaining fingers so that the two move together.

Sixth, verify that the green proceed LED is lit. If the red stop LED is lit and the buzzer sounds, the sample has less than 2 ppm SO₂ and there is no need to proceed.

Seventh, titrate the sample by adding the titrant solution dropwise from the syringe, being sure to note the starting point on the syringe. Try to accomplish the titration as rapidly as possible (in 2 minutes or less), but be careful near the endpoint so as not to overrun it. Be sure to maintain stirring or swirling throughout the entire procedure.

Eighth, during the titration, the red stop LED will briefly illuminate and the buzzer will sound transiently. These transient indicators will last longer and longer as you approach the endpoint, just as starch indicator does during a conventional titration. Take the endpoint as the first addition of titrant solution that causes the red stop LED and buzzer to stay on, for longer than 15 seconds. It is important to maintain stirring or swirling to detect the endpoint well. Read the endpoint volume off of the syringe.

Ninth, the free SO₂ content is calculated as

ppm SO₂=64*V*N*1000/(2*v)

where V=volume (mL) of titrant solution needed to reach the endpoint, N=normality of the titrant solution, v=volume (mL) of sample. With a 25 mL sample volume and titrant solution concentration of 0.0156 N, the calculation is simply

ppm SO₂=20*V

To analyze total sulfur dioxide, proceed as above except that after placing precisely 25 mL of wine or must in the titration vessel, one should add approximately 10 mL of the base reagent to the sample. Let stand approximately 10 minutes. Next, instead of adding approximately 2 mL acid reagent, add 9 mL acid reagent. The remaining steps are the same, except that the result calculated is the total SO₂ instead of the free SO₂.

In another procedure, a titrant solution may be standardized by using a known quantity of ascorbic acid. This procedure will determine or verify the concentration of the titrant solution.

First, accurately weigh out about 20 mg ascorbic acid. Dissolve it completely in precisely 100 mL of deionized water in a clean glass container.

Second, the actual concentration of ascorbic acid in this solution, C, is

C(meq/mL)=m/(88*W)

where m is the mass (mg) of the ascorbic acid and W is the volume (mL) of water, i.e., 100 mL, used to dissolve it.

Third, place precisely 25 mL of this solution into the titration vessel and proceed as for determining free SO₂. After determine the volume of titrant solution V (mL) needed to reach the endpoint, the normality of the titrant solution is calculated as

N(meq/mL)=v*C/V

where v is the volume of ascorbate solution (i.e., 25.0 mL), C is its concentration, and V is the volume (mL) of titrant solution required to reach the endpoint.

It should be emphasized that the above-described embodiments of the invention are merely possible examples of implementations of the invention. Many variations and modifications may be made to the above-described embodiments. All such modifications and variations are intended to be included herein within the scope of this disclosure and protected by the following claims.

1. A method of determining sulfite or related species concentration in an opaque solution comprising:
placing a measured amount of the opaque solution into a container,
contacting the measured opaque solution with an electrode comprising two poles, the electrode being electrically connected to a device for measuring current between the two poles,
adding a substance to make the measured opaque solution acidic,
adding iodide to make the measured opaque solution have an iodide concentration of greater than about 1 millimolar,
titrating the measured opaque solution with a standardized solution of an oxidant to an endpoint determined by a current between the poles above a predetermined threshold, wherein the device indicates the endpoint has been reached.

2. The method of claim 1 wherein the opaque solution is wine, must, beer, wort or juice.

3. The method of claim 1 further comprising:
calculating the concentration of sulfite or related species in the opaque solution.

4. (canceled)

5. The method of claim 1 wherein the minimum spacing of the portion of the poles in contact with the measured opaque solution is less than about 1 cm.

6. The method of claim 1 wherein the device impresses a voltage between the poles of less than about 0.5 V.

7. The method of claim 6 wherein the device impresses a voltage between the poles of about 0.1 V.

8. The method of claim 1 wherein the pH of the measured opaque solution is less than 2 after adding the substance.

9. The method of claim 1 wherein the substance is a liquid with a pH less than 2.

10. (canceled)

11. The method of claim 1 wherein the oxidant comprises iodine.

12. The method of claim 1 wherein the standardized solution comprises a solution of iodate.

13. The method of claim 1 wherein the predetermined threshold is equivalent to about 50 nA between the poles.

14. The method of claim 1 wherein the predetermined threshold is equivalent to about 1 micromolar iodine.

15. A method of determining sulfite or related species concentration in a fluid substance comprising:
creating a test fluid by adding to a known amount of the fluid substance
a first substance to make the test fluid acidic, and
a second substance to make the iodide concentration of the test fluid greater than about 1 millimolar;
contacting the known amount of fluid substance with an electrode comprising two poles, wherein the electrode is electrically connected to a device that can sense current between the two poles; and
adding a titrant, comprising iodine or iodate of a known concentration, to the test fluid until the device senses a current above a predetermined threshold.

16. The method of claim 15 wherein the fluid substance is wine, must, beer, wort or juice.

17. The method of claim 15 wherein the fluid substance substantially absorbs light at some visible wavelengths.

18. The method of claim 15, further comprising:
measuring the amount of titrant expended to cause the current to reach the predetermined threshold; and
determining the sulfite or related species concentration from the known amount of fluid substance, the known concentration of iodine or iodate in the titrant and the amount of titrant expended.

19. (canceled)

20. The method of claim 15 wherein the minimum spacing of the portion of the poles in contact with the known amount of fluid substance is less than about 1 cm.

21. The method of claim 15 wherein the device impresses a voltage between the poles of less than about 0.5 V.

22. The method of claim 21 wherein the device impresses a voltage between the poles of about 0.1 V.

23. The method of claim 15 wherein the first substance is a liquid with a pH less than 2.

24. (canceled)

25. The method of claim 15 wherein the predetermined threshold is equivalent to about 50 nA between the poles.

26. The method of claim 15 wherein the predetermined threshold is equivalent to about 1 micromolar iodine.

27. A method of measuring sulfite or related species in a fluid sample, comprising:
acidifying a known volume of fluid sample;
contacting the known volume of fluid sample with an electrode comprising two poles, wherein the electrode is electrically connected to a device that impresses a voltage of less than about 0.5 V between the two poles and senses the current between the two poles;
adding a titrant dropwise to the acidified fluid sample, wherein the titrant comprises iodine or iodate of a known concentration; and
ceasing addition of the titrant when the device indicates a current between the two poles above a predetermined threshold.

28. The method of claim 27 wherein the fluid substance is wine, must, beer, wort or juice.

29. The method of claim 27 wherein the fluid substance substantially absorbs light at some visible wavelengths.

30. The method of claim 27, further comprising:
determining the volume of titrant expended to cause the current to reach the predetermined threshold; and
determining the sulfite or related species concentration from the known volume of fluid sample, the known concentration of iodine or iodate in the titrant and the volume of titrant expended.

31. (canceled)

32. The method of claim 27 wherein the device impresses a voltage between the poles of about 0.1 V.

33. (canceled)
